(19) United States
(12) Patent Application Publication
(10) Pub. No.: US 2023/0048713 A1
(43) Pub. Date: Feb. 16, 2023

(54) WEARABLE TORSO SUPPORT DEVICE

(71) Applicant: Jamal Gibson, Haymarket, VA (US)

(72) Inventor: Jamal Gibson, Haymarket, VA (US)

(21) Appl. No.: 17/402,529

(22) Filed: Aug. 14, 2021

(51) Int. Cl. A61F 5/02; A61H 11/00

(52) U.S. Cl. CPC A61F 5/026; A61F 5/028; A61H 11/00; A61H 2201/1626; A61H 2201/1652; A61H 2201/5043; A61H 2201/0207; A61H 2201/0214; A61H 2201/0221

(57) ABSTRACT

A wearable torso support device, including a main body to removably connect around a pelvis of a user, including a front section to receive and support a stomach of the user thereon, a rear section disposed on at least a portion of each end of the front section to support at least a portion of a back of the user, and a plurality of body fasteners removably connected to at least a portion of at least one of the front section and the rear section, and a shoulder strap assembly to suspend the main body from shoulders of the user while being worn, the shoulder strap assembly including a first shoulder strap disposed on at least a portion of the main body to removably connect to at least one of the plurality of body fasteners and is adjustable in length based on a movement of the first shoulder strap, and a second shoulder strap disposed on at least a portion of the main body to removably connect to another at least one of the plurality of body fasteners and is adjustable in length based on a movement of the second shoulder strap.

### BACKGROUND

### 1. Field

The present general inventive concept relates generally to upper body support, and particularly, to a wearable torso support device.

### 2. Description of the Related Art

Some people may experience an increase in body weight due for a variety of reasons, such as overeating, genetics, lack of exercise, and/or pregnancy.

Unfortunately, the increase in weight causes people to experience stomach and/or back pain. Consequently, the increase in body weight provides a strain on the entire body as a whole and it becomes difficult for a person maintain correct posture. For example, other areas of the body that may be affected include legs, ankles, and/or feet. The increase in body weight may cause swelling that can lead to other medical complications.

Current support devices that are worn on a body of the person are limited to covering only an abdominal area and/or a back. However, these support devices do not alleviate the strain on the body from the increase in weight.

Therefore, there is a need for a wearable torso support device that supports a stomach and/or the back of the user by redistributing weight to different parts of the body.

### SUMMARY

The present general inventive concept provides a wearable torso support device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a wearable torso support device, including a main body to removably connect around a pelvis of a user, including a front section to receive and support a stomach of the user thereon, a rear section disposed on at least a portion of each end of the front section to support at least a portion of a back of the user, and a plurality of body fasteners removably connected to at least a portion of at least one of the front section and the rear section, and a shoulder strap assembly to suspend the main body from shoulders of the user while being worn, the shoulder strap assembly including a first shoulder strap disposed on at least a portion of the main body to removably connect to at least one of the plurality of body fasteners and is adjustable in length based on a movement of the first shoulder strap, and a second shoulder strap disposed on at least a portion of the main body to removably connect to another at least one of the plurality of body fasteners and is adjustable in length based on a movement of the second shoulder strap.

The first shoulder strap and the second shoulder strap each may include a first section to removably connect to at least one of the plurality of body fasteners, a shoulder strap fastener disposed on at least a portion of the first section to increase a length of the first section in response to moving the first section through the shoulder strap fastener in a first direction, and decrease the length of the first section in response to moving the first section through the shoulder strap fastener in a second direction opposite with respect to the first direction, and a second section disposed on at least a portion of the main body and connected to the first section.

The wearable torso support device may further include a tension body disposed between the first section and the second section to create a tension level on the shoulder strap assembly while worn, such that the tension body forces the shoulder strap assembly to move toward the tension body.

The wearable torso support device may further include a lumbar support padding disposed within at least a portion of the rear section to prevent the back of the user from bending while the rear section is connected to the back of the user.

The lumbar support padding may make the rear section more rigid than the front section.

The wearable torso support device may further include a packet receiving compartment disposed on at least a portion of the main body to receive at least one of a heat pack and a cool pack therein, such that the packet receiving compartment increases a temperature level or decreases the temperature level of the main body.

The wearable torso support device may further include a pressure dissemination unit disposed within at least a portion of the main body to simulate a massage.

The wearable torso support device may further include a temperature adjustment unit disposed within at least a portion of the main body to increase a temperature level or decrease the temperature level of the main body using at least one of a mesh of heating coils, a condenser, a fan, and a refrigerant to adjust the temperature level of the main body.

The wearable torso support device may further include a control unit comprising a processing unit running a program thereon to determine the temperature level of the temperature adjustment unit.

The wearable torso support device may further include an input unit comprising at least one button, the input unit disposed on at least a portion of the shoulder strap assembly to adjust the temperature level of the temperature adjustment unit using the control unit in response to depressing at least one button of the input unit.

The processing unit may save at least one preferred temperature level in response to depressing the at least one button for a predetermined amount of time.

The processing unit may automatically set the temperature adjustment unit to adjust the temperature level to at least one preferred temperature level.

The wearable torso support device may further include an external controller removably connected to at least a portion of the main body to transmit a command to the control unit to adjust the temperature level of the temperature adjustment unit in response to the external controller receiving at least one input.

The external controller may include a display unit to display an image of the rear section and activate at least one portion of the temperature adjustment unit corresponding to the portions of the rear section that have been selected on the display unit.

The foregoing and/or other features and utilities of the present general inventive concept may also be achieved by providing a wearable torso support device, including a main body to removably connect around a pelvis of a user and support a stomach of the user thereon, a shoulder strap assembly disposed on at least a portion of the main body to suspend the main body from shoulders of the user while being worn, such that the shoulder strap assembly is adjustable in length using a ratchet, a packet receiving compartment disposed on at least a portion of the main body to receive at least one of a heat pack and a cool pack therein, such that the packet receiving compartment increases a temperature level or decreases the temperature level of the main body, and a temperature adjustment unit disposed within at least a portion of the main body to increase a temperature level or decrease the temperature level of the main body using at least one of a mesh of heating coils, a condenser, a fan, and a refrigerant to adjust the temperature level of the main body.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a front perspective view of a wearable torso support device, according to an exemplary embodiment of the present general inventive concept;

FIG. 1B illustrates a rear perspective view of the wearable torso support device, according to an exemplary embodiment of the present general inventive concept;

FIG. 2A illustrates a front perspective view of a wearable torso support device, according to another exemplary embodiment of the present general inventive concept;

FIG. 2B illustrates a front perspective view of a wearable torso support device, according to another exemplary embodiment of the present general inventive concept;

FIG. 3 illustrates a front perspective view of a wearable torso support device, according to another exemplary embodiment of the present general inventive concept;

FIG. 4 illustrates a side perspective view of the wearable torso device as disposed on a user, according to an exemplary embodiment of the present general inventive concept; and

FIG. 5 illustrates a front perspective view of a wearable torso support device, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Wearable Torso Support Device 100
Main Body 110
Front Section 111
Rear Section 112
Body Fasteners 113
Shoulder Strap Assembly 120
First Shoulder Strap 121
First Section 121a
First Shoulder Strap Fastener 121b
Second Section 121c
Rear Section Fastener 121d
Second Shoulder Strap 122
First Section 122a
Second Shoulder Strap Fastener 122b
Second Section 122c
Rear Section Fastener 122d
Tension Body 130
Lumbar Support Padding 140
Packet Receiving Compartment 150
Pressure Dissemination Unit 160
Power Button 170
Power Source 180
Wearable Torso Support Device 200
Main Body 210
Front Section 211
Rear Section 212
Body Fasteners 213
Shoulder Strap Assembly 220
First Shoulder Strap 221
First Section 221a
First Shoulder Strap Fastener 221b
Second Section 221c
Second Shoulder Strap 222
First Section 222a
Second Shoulder Strap Fastener 222b
Second Section 222c
Tension Body 230
Lumbar Support Padding 240
Temperature Adjustment Unit 250
Control Unit 260
Processing Unit 261
Communication Unit 262
Storage Unit 263
Input Unit 270
Power Button 271
Heating Button 272
Cooling Button 273
Power Source 280
Wearable Torso Support Device 300
Main Body 310
Front Section 311
Rear Section 312
Body Fasteners 313
Shoulder Strap Assembly 320
First Shoulder Strap 321
First Section 321a
First Shoulder Strap Fastener 321b
Second Section 321c
Second Shoulder Strap 322
First Section 322a
Second Shoulder Strap Fastener 322b
Second Section 322c
Tension Body 330
Lumbar Support Padding 340
Temperature Adjustment Unit 350
Control Unit 360
Processing Unit 361
Communication Unit 362
Storage Unit 363
External Controller 370
Power Source 380
Wearable Torso Support Device 400
Main Body 410
Front Section 411
Rear Section 412
Body Fasteners 413
Shoulder Strap Assembly 420
First Shoulder Strap 421
First Section 421a
First Shoulder Strap Fastener 421b
Second Section 421c
Second Shoulder Strap 422
First Section 422a
Second Shoulder Strap Fastener 422b
Second Section 422c
Tension Body 430
Lumbar Support Padding 440
Packet Receiving Compartment 450
Temperature Adjustment Unit 460
Control Unit 470
Processing Unit 471
Communication Unit 472
Storage Unit 473
Input Unit 480
Power Button 481
Heating Button 482
Cooling Button 483
Power Source 490
Wearable Torso Support Device 500
Main Body 510
Front Section 511
First Body Fastener 511a
Second Body Fastener 511b
Rear Section 512
Shoulder Strap Assembly 520
First Shoulder Strap 521
First Section 521a
First Shoulder Strap Fastener 521b
Second Section 521c
Second Shoulder Strap 522
First Section 522a
Second Shoulder Strap Fastener 522b
Second Section 522c
Tension Assembly 530
First Tension Body 531
Second Tension Body 532
Body Connecting Strap 533
Shoulder Strap Connector Assembly 540
First Front Strap 541
Second Front Strap 542
First Rear Strap 543
Second Rear Strap 544
Lumbar Support Padding 550
Packet Receiving Compartment 560
Pressure Dissemination Unit 570
Power Button 580
Power Source 590

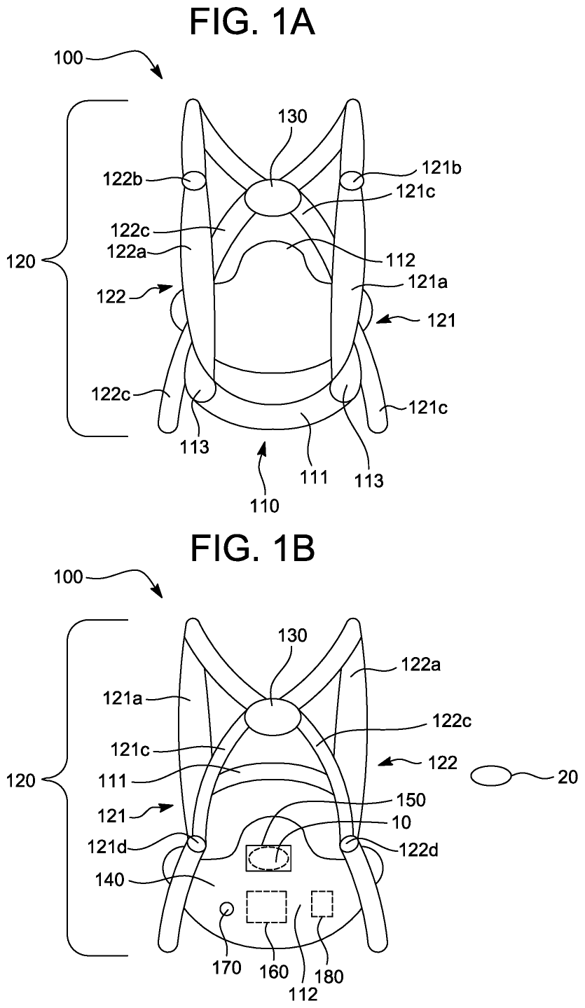

FIG. 1A illustrates a front perspective view of a wearable torso support device 100, according to an exemplary embodiment of the present general inventive concept.

FIG. 1B illustrates a rear perspective view of the wearable torso support device 100, according to an exemplary embodiment of the present general inventive concept.

The wearable torso support device 100 may be constructed from at least one of fabric, cloth, plastic, metal, and rubber, etc., but is not limited thereto.

Referring to FIGS. 1A and 1B, the wearable torso support device 100 may include a main body 110, a shoulder strap assembly 120, a tension body 130, a lumbar support padding 140, a packet receiving compartment 150, a pressure dissemination unit 160, a power button 170, and a power source 180, but is not limited thereto.

Referring again to FIGS. 1A and 1B, the main body 110 is illustrated to be a waist strap. However, the main body 110 may be a belt, a cord, a back brace, a chain, and/or any other type of waist worn accessory known to one of ordinary skill in the art.

Moreover, the main body 110 may be removably connected around a waist and/or a pelvis of a user 30. Moreover, the main body 110 may be worn around the waist and/or the pelvis to cover the waist and/or the pelvis, and support an abdomen and/or a stomach of the user 30. Also, the main body 110 may be constructed of various sizes (e.g., small, medium, large) based on a preference of the user 30. Alternatively, the main body 110 may be a one-size fits all.

The main body 110 may include a front section 111, a rear section 112, and a plurality of body fasteners 113, but is not limited thereto.

The front section 111 may be constructed of jersey fabric and/or elastane, but is not limited thereto. The front section 111 may at least partially deform (i.e., stretch, expand, bend) in response to receiving at least a portion of the abdomen and/or the stomach thereon. Additionally, the front section 111 may cover the stomach of the user 30. Also, the front section 111 may prevent the abdomen and/or the stomach from sagging (e.g., dropping toward a ground surface), such that the front section 111 may support the abdomen and/or the stomach of the user 30 while providing comfort.

The rear section 112 may be disposed on at least a portion of each end of the front section 111. In other words, the rear section 112 may comprise half of a size of the main body 110 and the front section 111 may comprise half of the size of the main body 110. The rear section 112 may cover at least a portion of a back of the user 30.

Each of the plurality of body fasteners 113 may include a clip, a clamp, a clasp, a hook, a ratchet, a magnet, and/or any combination thereof, but is not limited thereto.

The plurality of body fasteners 113 may be removably connected to at least a portion of the front section 111 and/or the rear section 112.

The shoulder strap assembly 120 may include a first shoulder strap 121 and a second shoulder strap 122, but is not limited thereto.

The first shoulder strap 121 may include a first section 121a, a first shoulder strap fastener 121b, a second section 121c, and a rear section fastener 121d, but is not limited thereto.

The first section 121a of the first shoulder strap 121 may be removably connected to at least a portion of the main body 110. More specifically, a first end of the first section 121a may removably connect to at least one of the plurality of body fasteners 113, such that at least one of the plurality of body fasteners 113 may secure (i.e. lock) the first end of the first section 121a thereto and/or release (i.e., unlock) the first end of the first section 121a therefrom.

Referring again to FIG. 1A, the first shoulder strap fastener 121b is illustrated to be a buckle. However, the first shoulder strap fastener 121b may be hooks and loops, a twine, a string, a rope, a magnet, a clip, a clamp, a clasp, a hook, and/or any combination thereof, but is not limited thereto.

The first shoulder strap fastener 121b may adjust a length of the first section 121a, such as increasing the length of the first section 121a by moving the first section 121a through the first shoulder strap fastener 121b in a first direction (e.g., toward the front section 111), and decreasing the length of the first section 121a by moving the first section 121a through the first shoulder strap fastener 121b in a second direction (e.g., away from the front section 111) opposite with respect to the first direction.

The second section 121c of the first shoulder strap 121 may be disposed on at least a portion of the main body 110. More specifically, a first end of the second section 121c may be disposed on at least a portion of the rear section 112.

Referring again to FIG. 1B, the rear section fastener 121d is illustrated to be a buckle. However, the rear section fastener 121d may be hooks and loops, a twine, a string, a rope, a magnet, a clip, a clamp, a clasp, a hook, and/or any combination thereof, but is not limited thereto.

The rear section fastener 121d may be disposed on at least a portion of the first end of the second section 121c. The rear section fastener 121d may adjust a length of the second section 121c, such as increasing the length of the second section 121c by moving the second section 121c through the rear section fastener 121d in a first direction (e.g., toward the rear section 112), and decreasing the length of the second section 121c by moving the second section 121c through the rear section fastener 121d in a second direction (e.g., away from the rear section 112) opposite with respect to the first direction.

The second shoulder strap 122 may include a first section 122a, a second shoulder strap fastener 122b, a second section 122c, and a rear section fastener 122d, but is not limited thereto.

The first section 122a of the second shoulder strap 122 may be removably connected to at least a portion of the main body 110. More specifically, a first end of the first section 122a may removably connect to at least one of the plurality of body fasteners 113, such that at least one of the plurality of body fasteners 113 may secure (i.e. lock) the first end of the first section 122a thereto and/or release (i.e., unlock) the first end of the first section 122a therefrom.

Referring again to FIG. 1A, the second shoulder strap fastener 122b is illustrated to be a buckle. However, the second shoulder strap fastener 122b may be hooks and loops, a twine, a string, a rope, a magnet, a clip, a clamp, a clasp, a hook, and/or any combination thereof, but is not limited thereto.

The second shoulder strap fastener 122b may adjust a length of the first section 122a, such as increasing the length of the first section 122a by moving the first section 122a through the second shoulder strap fastener 122b in a first direction (e.g., toward the front section 111), and decreasing the length of the first section 122a by moving the first section 122a through the second shoulder strap fastener 122b in a second direction (e.g., away from the front section 111) opposite with respect to the first direction.

The second section 122c of the second shoulder strap 122 may be disposed on at least a portion of the main body 110. More specifically, a first end of the second section 122c may be disposed on at least a portion of the rear section 112.

Referring again to FIG. 1B, the rear section fastener 122d is illustrated to be a buckle. However, the rear section fastener 122d may be hooks and loops, a twine, a string, a rope, a magnet, a clip, a clamp, a clasp, a hook, and/or any combination thereof, but is not limited thereto.

The rear section fastener 122d may be disposed on at least a portion of the first end of the second section 122c. The rear section fastener 122d may adjust a length of the second section 122c, such as increasing the length of the second section 122c by moving the second section 122c through the rear section fastener 122d in a first direction (e.g., toward the rear section 112), and decreasing the length of the second section 122c by moving the second section 122c through the rear section fastener 122d in a second direction (e.g., away from the rear section 112) opposite with respect to the first direction.

As such, the shoulder strap assembly 120 may by adjusted based on the preference of the user 30. Also, the shoulder strap assembly 120 may be connected to shoulders of the user 30, such that the main body 110 may be suspended from the shoulder strap assembly 120 while being worn by the user 30.

The tension body 130 may be constructed of jersey fabric and/or elastane, but is not limited thereto.

The tension body 130 may be disposed on at least a portion of a second end of the first section 121a, a second end of the second section 121c, a second end of the first section 122a, and/or a second end of the second section 122c. More specifically, the tension body 130 may be disposed between the second end of the first section 121a and/or the second end of the second section 121c. Similarly, the tension body 130 may be disposed between the second end of the first section 122a and/or the second end of the second section 122c. Moreover, the first section 121a, the second section 121c, the first section 122a, and/or the second section 122c may form a cross section (e.g., X-shape) at the tension body 130, such that the cross section increases support with respect to a front portion of the first section 121a and/or the first section 122a.

Furthermore, the tension body 130 may have a predetermined tension level to pull the first section 121a, the second section 121c, the first section 122a, and/or the second section 122c toward the tension body 130. In other words, the tension body 130 may be constructed to create a tension level on the shoulder strap assembly 120 while worn, such that the tension body 130 improves a posture of the user 30 while wearing the shoulder strap assembly 120 by forcing the shoulder strap assembly 120 to move toward the tension body 130. Accordingly, the tension body 130 may force the posture of the user 30 to be straight instead of slouched and/or bending forward toward the stomach.

The lumbar support padding 140 may be constructed of foam and/or a gel cushion, but is not limited thereto.

The lumbar support padding 140 may be disposed within at least a portion of the rear section 112. The lumbar support padding 140 may be firmer and/or more rigid, such that the rear section 112 may be firmer and/or more rigid with respect to the front section 111. As such, the lumbar support padding 140 may prevent the back of the user 30 from bending while the rear section 112 is connected to the back of the user 30.

The packet receiving compartment 150 may be constructed of polyethylene terephthalate (PET), closed-cell extruded polystyrene foam, polyurethane, polypropylene, and/or refrigerant gel, but is not limited thereto.

The packet receiving compartment 150 may be disposed on at least a portion of an outer surface of the rear section 112 and/or an inner surface of the rear section 112. The packet receiving compartment 150 may receive a heat pack 10 and/or a cool pack 20 therein. Thus, the packet receiving compartment 150 may be insulated and designed to retain heat from the heat pack 10 and/or cool air from the cold pack 20 therein. Accordingly, the packet receiving compartment 150 may comfort the back of the user 30 in response to dissipating a hot temperature (i.e., increasing a temperature level) and/or a cold temperature (i.e., decreasing the temperature level) on the outer surface and/or the inner surface of the rear section 112.

The pressure dissemination unit 160 may be disposed within at least a portion of the rear section 112. The pressure dissemination unit 160 may include various devices to simulate a massage, including, but not limited to, vibration devices, pistons, mechanical rollers, compression devices, stretching devices, kneading devices, pressing devices, oscillators, gears, etc. As such, the pressure dissemination unit 160 may provide a pressured sensation to the back of the user 30, thus simulating a massage.

The power button 170 may be disposed on at least a portion of the rear section 112 and connected to the pressure dissemination unit 160. The pressure dissemination unit 160 may turn on in response to depressing the power button 170 a first time. Conversely, the pressure dissemination unit 160 may turn off in response to depressing the power button 170 a second time.

Alternatively, the power button 170 may be a control panel 170 to change at least one operation of the pressure dissemination unit 160, such as a speed, pressure intensity, or pre-programmed massage style, such as Swedish, deep tissue, shiatsu, Thai, simulation of fingers, elbows, hands chopping, vibration, reiki, reflexology, and/or pounding.

The power source 180 may include a battery (e.g., alkaline, nickel cadmium, lithium ion) and a solar cell, but is not limited thereto.

The power source 180 may be disposed within at least a portion of the rear section 112. The power source 180 may send power to the pressure dissemination unit 160 and/or the power button 170.

Therefore, the wearable torso support device 100 may improve posture of the user 30 and/or prevent back pain due to being overweight and/or a pregnancy. Also, the wearable torso support device 100 may alleviate strain on the legs, ankles, and/or feet of the user 30.

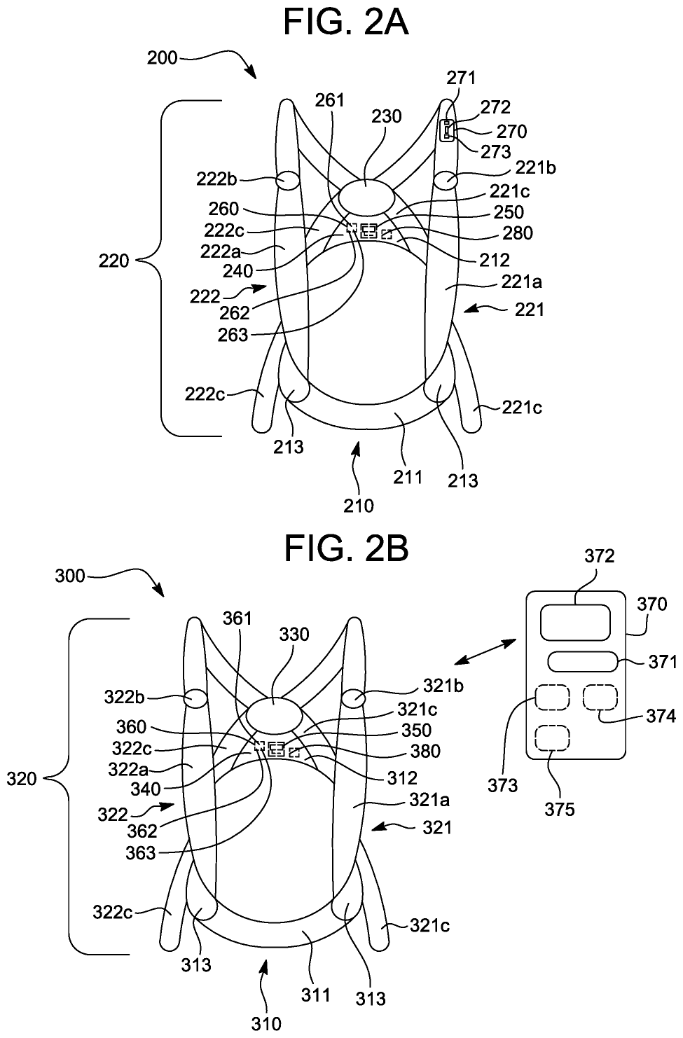

FIG. 2A illustrates a front perspective view of a wearable torso support device 200, according to another exemplary embodiment of the present general inventive concept.

The wearable torso support device 200 may be constructed from at least one of fabric, cloth, plastic, metal, and rubber, etc., but is not limited thereto.

The wearable torso support device 200 may include a main body 210, a shoulder strap assembly 220, a tension body 230, a lumbar support padding 240, a temperature adjustment unit 250, a control unit 260, an input unit 270, and a power source 280, but is not limited thereto.

Referring to FIG. 2A, the main body 210 is illustrated to be a waist strap. However, the main body 210 may be a belt, a cord, a back brace, a chain, and/or any other type of waist worn accessory known to one of ordinary skill in the art. Moreover, the main body 210 may be removably connected around a waist and/or a pelvis of a user 30. Moreover, the main body 210 may be worn around the waist and/or the pelvis to cover the waist and/or the pelvis, and support an abdomen and/or a stomach of the user 30. Also, the main body 210 may be constructed of various sizes (e.g., small, medium, large) based on a preference of the user 30. Alternatively, the main body 210 may be a one-size fits all.

The main body 210 may include a front section 211, a rear section 212, and a plurality of body fasteners 213, but is not limited thereto.

The front section 211 may be constructed of jersey fabric and/or elastane, but is not limited thereto. The front section 211 may at least partially deform (i.e., stretch, expand, bend) in response to receiving at least a portion of the abdomen and/or the stomach thereon. Additionally, the front section 211 may cover the stomach of the user 30. Also, the front section 211 may prevent the abdomen and/or the stomach from sagging (e.g., dropping toward a ground surface), such that the front section 211 may support the abdomen and/or the stomach of the user 30 while providing comfort.

The rear section 212 may be disposed on at least a portion of each end of the front section 211. In other words, the rear section 212 may comprise half of a size of the main body 210 and the front section 211 may comprise half of the size of the main body 210. The rear section 212 may cover at least a portion of a back of the user 30.

Each of the plurality of body fasteners 213 may include a clip, a clamp, a clasp, a hook, a ratchet, a magnet, and/or any combination thereof, but is not limited thereto.

The plurality of body fasteners 213 may be removably connected to at least a portion of the front section 211 and/or the rear section 212.

The shoulder strap assembly 220 may include a first shoulder strap 221 and a second shoulder strap 222, but is not limited thereto.

The first shoulder strap 221 may include a first section 221a, a first shoulder strap fastener 221b, and a second section 221c, but is not limited thereto.

The first section 221a of the first shoulder strap 221 may be removably connected to at least a portion of the main body 210. More specifically, a first end of the first section 221a may removably connect to at least one of the plurality of body fasteners 213, such that at least one of the plurality of body fasteners 213 may secure (i.e. lock) the first end of the first section 221a thereto and/or release (i.e., unlock) the first end of the first section 221a therefrom.

Referring again to FIG. 2A, the first shoulder strap fastener 221b is illustrated to be a buckle. However, the first shoulder strap fastener 221b may be hooks and loops, a twine, a string, a rope, a magnet, a clip, a clamp, a clasp, a hook, and/or any combination thereof, but is not limited thereto.

The first shoulder strap fastener 221b may adjust a length of the first section 221a, such as increasing the length of the first section 221a by moving the first section 221a through the first shoulder strap fastener 221b in a first direction (e.g., toward the front section 211), and decreasing the length of the first section 221a by moving the first section 221a through the first shoulder strap fastener 221b in a second direction (e.g., away from the front section 211) opposite with respect to the first direction.

The second section 221c of the first shoulder strap 221 may be disposed on at least a portion of the main body 210. More specifically, a first end of the second section 221c may be disposed on at least a portion of the rear section 212.

The second shoulder strap 222 may include a first section 222a, a second shoulder strap fastener 222b, and a second section 222c, but is not limited thereto.

The first section 222a of the second shoulder strap 222 may be removably connected to at least a portion of the main body 210. More specifically, a first end of the first section 222a may removably connect to at least one of the plurality of body fasteners 213, such that at least one of the plurality of body fasteners 213 may secure (i.e. lock) the first end of the first section 222a thereto and/or release (i.e., unlock) the first end of the first section 222a therefrom.

Referring again to FIG. 2A, the second shoulder strap fastener 222b is illustrated to be a buckle. However, the second shoulder strap fastener 222b may be hooks and loops, a twine, a string, a rope, a magnet, a clip, a clamp, a clasp, a hook, and/or any combination thereof, but is not limited thereto.

The second shoulder strap fastener 222b may adjust a length of the first section 222a, such as increasing the length of the first section 222a by moving the first section 222a through the second shoulder strap fastener 222b in a first direction (e.g., toward the front section 211), and decreasing the length of the first section 222a by moving the first section 222a through the second shoulder strap fastener 222b in a second direction (e.g., away from the front section 211) opposite with respect to the first direction.

The second section 222c of the second shoulder strap 222 may be disposed on at least a portion of the main body 210. More specifically, a first end of the second section 222c may be disposed on at least a portion of the rear section 212.

As such, the shoulder strap assembly 220 may by adjusted based on the preference of the user 30. Also, the shoulder strap assembly 220 may be connected to shoulders of the user 30, such that the main body 210 may be suspended from the shoulder strap assembly 220 while being worn by the user 30.

The tension body 230 may be constructed of jersey fabric and/or elastane, but is not limited thereto.

The tension body 230 may be disposed on at least a portion of a second end of the first section 221a, a second end of the second section 221c, a second end of the first section 222a, and/or a second end of the second section 222c. More specifically, the tension body 230 may be disposed between the second end of the first section 221a and/or the second end of the second section 221c. Similarly, the tension body 230 may be disposed between the second end of the first section 222a and/or the second end of the second section 222c. Moreover, the first section 221a, the second section 221c, the first section 222a, and/or the second section 222c may form a cross section (e.g., X-shape) at the tension body 230, such that the cross section increases support with respect to a front portion of the first section 221a and/or the first section 222a. Furthermore, the tension body 230 may have a predetermined tension level to pull the first section 221a, the second section 221c, the first section 222a, and/or the second section 222c toward the tension body 230. In other words, the tension body 230 may be constructed to create a tension level on the shoulder strap assembly 220 while worn, such that the tension body 230 improves a posture of the user 30 while wearing the shoulder strap assembly 220 by forcing the shoulder strap assembly 220 to move toward the tension body 230. Accordingly, the tension body 230 may force the posture of the user 30 to be straight instead of slouched and/or bending forward toward the stomach.

The lumbar support padding 240 may be constructed of foam and/or a gel cushion, but is not limited thereto.

The lumbar support padding 240 may be disposed within at least a portion of the rear section 212. The lumbar support padding 240 may be firmer and/or more rigid, such that the rear section 212 may be firmer and/or more rigid with respect to the front section 211. As such, the lumbar support padding 240 may prevent the back of the user 30 from bending while the rear section 212 is connected to the back of the user 30.

The temperature adjustment unit 250 may include a mesh of heating coils, a condenser, a fan, and a coolant/refrigerant, but is not limited thereto.

The temperature adjustment unit 250 may be disposed within at least a portion of the rear section 212. Thus, the temperature adjustment unit 250 may increase a temperature level and/or decrease a temperature level of a surface of the rear section 212. More specifically, the temperature adjustment unit 250 may generate heat from the mesh of heating coils and/or generate cold air from the condenser, the fan, and/or the coolant/refrigerant to adjust the temperature level of the surface of the rear section 212. Accordingly, the temperature adjustment unit 250 may comfort the back of the user 30 in response to dissipating a hot temperature (i.e., increasing the temperature level) and/or a cold temperature (i.e., decreasing the temperature level) on the outer surface and/or the inner surface of the rear section 212.

The control unit 260 may include a processing unit 261, a communication unit 262, and a storage unit 263, but is not limited thereto.

The processing unit 261 (or central processing unit, CPU) may include electronic circuitry to carry out instructions of a computer program by performing basic arithmetic, logical, control and input/output (I/O) operations specified by the instructions. The processing unit 261 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit 261 may also include a microprocessor and a microcontroller.

The communication unit 262 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), radio frequency (RF) communication, USB, global positioning system (GPS), Firewire, and Ethernet.

The storage unit 263 may include a random access memory (RAM), a read-only memory (ROM), a hard disk, a flash drive, a database connected to the Internet, cloud-based storage, Internet-based storage, or any other type of storage unit.

The storage unit 263 may be preprogrammed and/or store a software application and/or a mobile application to be executed using the processing unit 261. For ease of description, the software and/or the mobile application will be hereinafter referred to as an app.

The input unit 270 may include a power button 271, a heating button 272, and a cooling button 273, but is not limited thereto.

Although the input unit 270 is described as using a plurality of buttons, the input unit 270 may include a plurality of switches, a plurality of levers, a plurality of knobs, a plurality of knobs, and/or any combination thereof, but is not limited thereto.

The input unit 270 may be disposed on at least a portion of the main body 210 and/or the shoulder strap assembly 220 and connected to the temperature adjustment unit 250. For example, referring again to FIG. 2A, the input unit 270 may be disposed on at least a portion of the first section 221a of the first shoulder strap 221. As such, the input unit 270 may be accessible on a front portion of the user 30, such as a chest of the user 30 while wearing the shoulder strap assembly 220. The input unit 270 may correspond to an operation of the temperature adjustment unit 250.

More specifically, the temperature adjustment unit 250 may turn on in response to depressing the power button 271 a first time. Conversely, the temperature adjustment unit 250 may turn off in response to depressing the power button 271 a second time.

It is important to note that the processing unit 261 may communicate with the input unit 270 either through a direct wired connection and/or wirelessly through the communication unit 262, such as Bluetooth. The temperature adjustment unit 250 may increase the temperature level of the rear section 212 in response to depressing the heating button 272. Alternatively, the temperature adjustment unit 250 may decrease the temperature level of the rear section 212 in response to depressing the cooling button 273. Moreover, the processing unit 261 executing the app may determine various temperature levels based on the app in response to depressing the heating button 272 and/or the cooling button 273. Moreover, the processing unit 261 executing the app may save at least one first preferred temperature level of the user 30 in response to depressing the power button 271 and the heating button 272 simultaneously and/or for a predetermined amount of time. Alternatively, the processing unit 261 executing the app may save at least one second preferred temperature level of the user 30 in response to depressing the power button 271 and the cooling button 273 simultaneously and/or for the predetermined amount of time. As such, the processing unit 261 executing the app may automatically set the temperature adjustment unit 250 to adjust the temperature level of the rear section 212 to the at least one first preferred temperature level or the at least one second preferred temperature level after the temperature adjustment unit 250 is next turned on.

Furthermore, the processing unit 261 executing the app may cycle the temperature adjustment unit 250 through a variety of temperature levels in response to depressing the heating button 272 and the cooling button 273 simultaneously, such that the user 30 experiences both hot and/or cold temperatures.

The power source 280 may include a battery (e.g., alkaline, nickel cadmium, lithium ion) and a solar cell, but is not limited thereto.

The power source 280 may be disposed within at least a portion of the rear section 212. The power source 280 may send power to the temperature adjustment unit 250, the control unit 260, and/or the input unit 270.

Therefore, the wearable torso support device 200 may improve posture of the user 30 and/or prevent back pain due to being overweight and/or a pregnancy. Also, the wearable torso support device 200 may alleviate strain on the legs, ankles, and/or feet of the user 30.

FIG. 2B illustrates a front perspective view of a wearable torso support device 300, according to another exemplary embodiment of the present general inventive concept.

The wearable torso support device 300 may be constructed from at least one of fabric, cloth, plastic, metal, and rubber, etc., but is not limited thereto.

The wearable torso support device 300 may include a main body 310, a shoulder strap assembly 320, a tension body 330, a lumbar support padding 340, a temperature adjustment unit 350, a control unit 360, an external controller 370, and a power source 380, but is not limited thereto.

Referring to FIG. 2B, the main body 310 is illustrated to be a waist strap. However, the main body 310 may be a belt, a cord, a back brace, a chain, and/or any other type of waist worn accessory known to one of ordinary skill in the art. Moreover, the main body 310 may be removably connected around a waist and/or a pelvis of a user 30. Moreover, the main body 310 may be worn around the waist and/or the pelvis to cover the waist and/or the pelvis, and support an abdomen and/or a stomach of the user 30. Also, the main body 310 may be constructed of various sizes (e.g., small, medium, large) based on a preference of the user 30. Alternatively, the main body 310 may be a one-size fits all.

The main body 310 may include a front section 311, a rear section 312, and a plurality of body fasteners 313, but is not limited thereto.

The front section 311 may be constructed of jersey fabric and/or elastane, but is not limited thereto. The front section 311 may at least partially deform (i.e., stretch, expand, bend) in response to receiving at least a portion of the abdomen and/or the stomach thereon. Additionally, the front section 311 may cover the stomach of the user 30. Also, the front section 311 may prevent the abdomen and/or the stomach from sagging (e.g., dropping toward a ground surface), such that the front section 311 may support the abdomen and/or the stomach of the user 30 while providing comfort.

The rear section 312 may be disposed on at least a portion of each end of the front section 311. In other words, the rear section 312 may comprise half of a size of the main body 310 and the front section 311 may comprise half of the size of the main body 310. The rear section 312 may cover at least a portion of a back of the user 30.

Each of the plurality of body fasteners 313 may include a clip, a clamp, a clasp, a hook, a ratchet, a magnet, and/or any combination thereof, but is not limited thereto.

The plurality of body fasteners 313 may be removably connected to at least a portion of the front section 311 and/or the rear section 312.

The shoulder strap assembly 320 may include a first shoulder strap 321 and a second shoulder strap 322, but is not limited thereto.

The first shoulder strap 321 may include a first section 321a, a first shoulder strap fastener 321b, and a second section 321c, but is not limited thereto.

The first section 321a of the first shoulder strap 321 may be removably connected to at least a portion of the main body 310. More specifically, a first end of the first section 321a may removably connect to at least one of the plurality of body fasteners 313, such that at least one of the plurality of body fasteners 313 may secure (i.e. lock) the first end of the first section 321a thereto and/or release (i.e., unlock) the first end of the first section 321a therefrom.

Referring again to FIG. 2B, the first shoulder strap fastener 321b is illustrated to be a buckle. However, the first shoulder strap fastener 321b may be hooks and loops, a twine, a string, a rope, a magnet, a clip, a clamp, a clasp, a hook, and/or any combination thereof, but is not limited thereto.

The first shoulder strap fastener 321b may adjust a length of the first section 321a, such as increasing the length of the first section 321a by moving the first section 321a through the first shoulder strap fastener 321b in a first direction (e.g., toward the front section 311), and decreasing the length of the first section 321a by moving the first section 321a through the first shoulder strap fastener 321b in a second direction (e.g., away from the front section 311) opposite with respect to the first direction.

The second section 321c of the first shoulder strap 321 may be disposed on at least a portion of the main body 310. More specifically, a first end of the second section 321c may be disposed on at least a portion of the rear section 312.

The second shoulder strap 322 may include a first section 322a, a second shoulder strap fastener 322b, and a second section 322c, but is not limited thereto.

The first section 322a of the second shoulder strap 322 may be removably connected to at least a portion of the main body 310. More specifically, a first end of the first section 322a may removably connect to at least one of the plurality of body fasteners 313, such that at least one of the plurality of body fasteners 313 may secure (i.e. lock) the first end of the first section 322a thereto and/or release (i.e., unlock) the first end of the first section 322a therefrom.

Referring again to FIG. 2B, the second shoulder strap fastener 322b is illustrated to be a buckle. However, the second shoulder strap fastener 322b may be hooks and loops, a twine, a string, a rope, a magnet, a clip, a clamp, a clasp, a hook, and/or any combination thereof, but is not limited thereto.

The second shoulder strap fastener 322b may adjust a length of the first section 322a, such as increasing the length of the first section 322a by moving the first section 322a through the second shoulder strap fastener 322b in a first direction (e.g., toward the front section 311), and decreasing the length of the first section 322a by moving the first section 322a through the second shoulder strap fastener 322b in a second direction (e.g., away from the front section 311) opposite with respect to the first direction.

The second section 322c of the second shoulder strap 322 may be disposed on at least a portion of the main body 310. More specifically, a first end of the second section 322c may be disposed on at least a portion of the rear section 312.

As such, the shoulder strap assembly 320 may by adjusted based on the preference of the user 30. Also, the shoulder strap assembly 320 may be connected to shoulders of the user 30, such that the main body 310 may be suspended from the shoulder strap assembly 320 while being worn by the user 30.

The tension body 330 may be constructed of jersey fabric and/or elastane, but is not limited thereto.

The tension body 330 may be disposed on at least a portion of a second end of the first section 321a, a second end of the second section 321c, a second end of the first section 322a, and/or a second end of the second section 322c. More specifically, the tension body 330 may be disposed between the second end of the first section 321a and/or the second end of the second section 321c. Similarly, the tension body 330 may be disposed between the second end of the first section 322a and/or the second end of the second section 322c. Moreover, the first section 321a, the second section 321c, the first section 322a, and/or the second section 322c may form a cross section (e.g., X-shape) at the tension body 330, such that the cross section increases support with respect to a front portion of the first section 321a and/or the first section 322a. Furthermore, the tension body 330 may have a predetermined tension level to pull the first section 321a, the second section 321c, the first section 322a, and/or the second section 322c toward the tension body 330. In other words, the tension body 330 may be constructed to create a tension level on the shoulder strap assembly 320 while worn, such that the tension body 330 improves a posture of the user 30 while wearing the shoulder strap assembly 320 by forcing the shoulder strap assembly 320 to move toward the tension body 330. Accordingly, the tension body 330 may force the posture of the user 30 to be straight instead of slouched and/or bending forward toward the stomach.

The lumbar support padding 340 may be constructed of foam and/or a gel cushion, but is not limited thereto.

The lumbar support padding 340 may be disposed within at least a portion of the rear section 312. The lumbar support padding 340 may be firmer and/or more rigid, such that the rear section 312 may be firmer and/or more rigid with respect to the front section 311. As such, the lumbar support padding 240 may prevent the back of the user 30 from bending while the rear section 312 is connected to the back of the user 30.

The temperature adjustment unit 350 may include a mesh of heating coils, a condenser, a fan, and a coolant/refrigerant, but is not limited thereto.

The temperature adjustment unit 350 may be disposed within at least a portion of the rear section 312. Thus, the temperature adjustment unit 350 may increase a temperature level and/or decrease a temperature level of a surface of the rear section 312. More specifically, the temperature adjustment unit 350 may generate heat from the mesh of heating coils and/or generate cold air from the condenser, the fan, and/or the coolant/refrigerant to adjust the temperature level of the surface of the rear section 312. Accordingly, the temperature adjustment unit 350 may comfort the back of the user 30 in response to dissipating a hot temperature (i.e., increasing the temperature level) and/or a cold temperature (i.e., decreasing the temperature level) on the outer surface and/or the inner surface of the rear section 312.

The control unit 360 may include a processing unit 361, a communication unit 362, and a storage unit 363, but is not limited thereto.

The processing unit 361 (or central processing unit, CPU) may include electronic circuitry to carry out instructions of a computer program by performing basic arithmetic, logical, control and input/output (I/O) operations specified by the instructions. The processing unit 361 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit 361 may also include a microprocessor and a microcontroller.

The communication unit 362 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), radio frequency (RF) communication, USB, global positioning system (GPS), Firewire, and Ethernet.

The storage unit 363 may include a random access memory (RAM), a read-only memory (ROM), a hard disk, a flash drive, a database connected to the Internet, cloud-based storage, Internet-based storage, or any other type of storage unit.

The storage unit 363 may be preprogrammed and/or store a software application and/or a mobile application to be executed using the processing unit 361. For ease of description, the software and/or the mobile application will be hereinafter referred to as an app.

The external controller 370 may be a mobile device, a cell phone, a remote controller, a tablet computer, a personal digital assistant (PDA), and a watch, but is not limited thereto. Also, the external controller 370 may detachably connect to at least a portion of the main body 310 and/or the shoulder strap assembly 320.

The external controller 370 may include an input unit 371, a display unit 372, a processing unit 373, a communication unit 374, and a storage unit 375, but is not limited thereto.

The input unit 371 may include a keyboard, a touchpad, a mouse, a trackball, a stylus, a voice recognition unit, a visual data reader, a camera, a wireless device reader, a fingerprint reader, an iris scanner, a facial recognition unit, and a holographic input unit.

The display unit 372 may include a plasma screen, an LCD screen, a light emitting diode (LED) screen, an organic LED (OLED) screen, a computer monitor, a hologram output unit, a sound outputting unit, or any other type of device that visually or aurally displays data.

Also, the display unit 372 may be combined with the input unit 371 to be a touch-screen.

The processing unit 373 (or central processing unit, CPU) may include electronic circuitry to carry out instructions of a computer program by performing basic arithmetic, logical, control and input/output (I/O) operations specified by the instructions. The processing unit 373 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit 373 may also include a microprocessor and a microcontroller.

The communication unit 374 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The storage unit 375 may include a random access memory (RAM), a read-only memory (ROM), a hard disk, a flash drive, a database connected to the Internet, cloud-based storage, Internet-based storage, or any other type of storage unit.

The external controller 370 may access the Internet via the communication unit 374 to allow the user 30 to access a website, and/or may allow a mobile application and/or the software application to be executed using the processing unit 373. For ease of description, the mobile and/or the software application will be hereinafter referred to as an app. The app may be downloaded from the Internet to be stored on the storage unit 375. Also, the app stored on the storage unit 363 may be the same as the app stored on the storage unit 375 of the external controller 370.

The input unit 371 may receive an input corresponding to an operation of the temperature adjustment unit 350. More specifically, the temperature adjustment unit 350 may turn on in response to receiving a power on command from the input unit 371 a first time. Conversely, the temperature adjustment unit 350 may turn off in response to receiving a power off command from the input unit 371 a second time. Moreover, the processing unit 373 executing the app may transmit a command from the communication unit 374 to the communication unit 362 to turn on and/or turn off the temperature adjustment unit 350 based on the received input on the input unit 371.

The temperature adjustment unit 350 may increase the temperature level of the rear section 312 in response to the input unit 371 receiving a heat command. Alternatively, the temperature adjustment unit 350 may decrease the temperature level of the rear section 312 in response to the input unit 371 receiving a cool command. Moreover, the processing unit 371 executing the app may adjust the temperature level to various temperature levels based on commands received on the input unit 371.

The display unit 372 may display an image of the rear section 312, and the user 30 may use the input unit 371 to target portions of the rear section 312 as displayed on the display unit 372, to activate at least one portion of the temperature adjustment unit 350 corresponding to the portions of the rear section 312 that have been selected on the display unit 372.

Moreover, the processing unit 373 executing the app may save at least one first preferred temperature level of the user 30 in response to selecting a first save on the display unit 372. Alternatively, the processing unit 373 executing the app may save at least one second preferred temperature level of the user 30 in response to selecting a second save on the display unit 372. As such, the processing unit 261 executing the app may automatically set the temperature adjustment unit 250 to adjust the temperature level of the rear section 312 to the at least one first preferred temperature level or the at least one second preferred temperature level after the temperature adjustment unit 350 is next turned on.

Furthermore, the temperature adjustment unit 350 may cycle through a variety of temperature levels in response to the processing unit 373 receiving a temperature cycle command on the input unit 371, such that the user 30 experiences both hot and/or cold temperatures.

The power source 380 may include a battery (e.g., alkaline, nickel cadmium, lithium ion) and a solar cell, but is not limited thereto.

The power source 380 may be disposed within at least a portion of the rear section 212. The power source 380 may send power to the temperature adjustment unit 350 and/or the control unit 360.

Therefore, the wearable torso support device 300 may improve posture of the user 30 and/or prevent back pain due to being overweight and/or a pregnancy. Also, the wearable torso support device 300 may alleviate strain on the legs, ankles, and/or feet of the user 30.

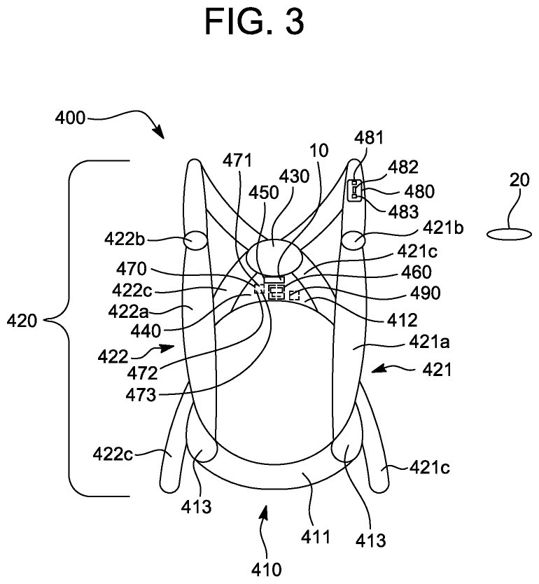

FIG. 3 illustrates a front perspective view of a wearable torso support device 400, according to another exemplary embodiment of the present general inventive concept.

The wearable torso support device 400 may be constructed from at least one of fabric, cloth, plastic, metal, and rubber, etc., but is not limited thereto.

The wearable torso support device 400 may include a main body 410, a shoulder strap assembly 420, a tension body 430, a lumbar support padding 440, a packet receiving compartment 450, a temperature adjustment unit 460, a control unit 470, an input unit 480, and a power source 490, but is not limited thereto.

Referring to FIG. 3, the main body 410 is illustrated to be a waist strap. However, the main body 410 may be a belt, a cord, a back brace, a chain, and/or any other type of waist worn accessory known to one of ordinary skill in the art. Moreover, the main body 410 may be removably connected around a waist and/or a pelvis of a user 30. Moreover, the main body 410 may be worn around the waist and/or the pelvis to cover the waist and/or the pelvis, and support an abdomen and/or a stomach of the user 30. Also, the main body 410 may be constructed of various sizes (e.g., small, medium, large) based on a preference of the user 30. Alternatively, the main body 410 may be a one-size fits all.

The main body 410 may include a front section 411, a rear section 412, and a plurality of body fasteners 413, but is not limited thereto.

The front section 411 may be constructed of jersey fabric and/or elastane, but is not limited thereto. The front section 411 may at least partially deform (i.e., stretch, expand, bend) in response to receiving at least a portion of the abdomen and/or the stomach thereon. Additionally, the front section 411 may cover the stomach of the user 30. Also, the front section 411 may prevent the abdomen and/or the stomach from sagging (e.g., dropping toward a ground surface), such that the front section 411 may support the abdomen and/or the stomach of the user 30 while providing comfort.

The rear section 412 may be disposed on at least a portion of each end of the front section 411. In other words, the rear section 412 may comprise half of a size of the main body 410 and the front section 411 may comprise half of the size of the main body 410. The rear section 412 may cover at least a portion of a back of the user 30.

Each of the plurality of body fasteners 413 may include a clip, a clamp, a clasp, a hook, a ratchet, a magnet, and/or any combination thereof, but is not limited thereto.

The plurality of body fasteners 413 may be removably connected to at least a portion of the front section 411 and/or the rear section 412.

The shoulder strap assembly 420 may include a first shoulder strap 421 and a second shoulder strap 422, but is not limited thereto.

The first shoulder strap 421 may include a first section 421a, a first shoulder strap fastener 421b, and a second section 421c, but is not limited thereto.

The first section 421a of the first shoulder strap 421 may be removably connected to at least a portion of the main body 410. More specifically, a first end of the first section 421a may removably connect to at least one of the plurality of body fasteners 413, such that at least one of the plurality of body fasteners 413 may secure (i.e. lock) the first end of the first section 421a thereto and/or release (i.e., unlock) the first end of the first section 421a therefrom.

Referring again to FIG. 3, the first shoulder strap fastener 421b is illustrated to be a buckle. However, the first shoulder strap fastener 421b may be hooks and loops, a twine, a string, a rope, a magnet, a clip, a clamp, a clasp, a hook, and/or any combination thereof, but is not limited thereto.

The first shoulder strap fastener 421b may adjust a length of the first section 421a, such as increasing the length of the first section 421a by moving the first section 421a through the first shoulder strap fastener 421b in a first direction (e.g., toward the front section 411), and decreasing the length of the first section 421a by moving the first section 421a through the first shoulder strap fastener 421b in a second direction (e.g., away from the front section 411) opposite with respect to the first direction.

The second section 421c of the first shoulder strap 421 may be disposed on at least a portion of the main body 410. More specifically, a first end of the second section 421c may be disposed on at least a portion of the rear section 412.

The second shoulder strap 422 may include a first section 422a, a second shoulder strap fastener 422b, and a second section 422c, but is not limited thereto.

The first section 422a of the second shoulder strap 422 may be removably connected to at least a portion of the main body 410. More specifically, a first end of the first section 422a may removably connect to at least one of the plurality of body fasteners 413, such that at least one of the plurality of body fasteners 413 may secure (i.e. lock) the first end of the first section 422a thereto and/or release (i.e., unlock) the first end of the first section 422a therefrom.

Referring again to FIG. 3, the second shoulder strap fastener 422b is illustrated to be a buckle. However, the second shoulder strap fastener 422b may be hooks and loops, a twine, a string, a rope, a magnet, a clip, a clamp, a clasp, a hook, and/or any combination thereof, but is not limited thereto.

The second shoulder strap fastener 422b may adjust a length of the first section 422a, such as increasing the length of the first section 422a by moving the first section 422a through the second shoulder strap fastener 422b in a first direction (e.g., toward the front section 411), and decreasing the length of the first section 422a by moving the first section 422a through the second shoulder strap fastener 422b in a second direction (e.g., away from the front section 411) opposite with respect to the first direction.

The second section 422c of the second shoulder strap 422 may be disposed on at least a portion of the main body 410. More specifically, a first end of the second section 422c may be disposed on at least a portion of the rear section 412.

As such, the shoulder strap assembly 420 may by adjusted based on the preference of the user 30. Also, the shoulder strap assembly 420 may be connected to shoulders of the user 30, such that the main body 410 may be suspended from the shoulder strap assembly 420 while being worn by the user 30.

The tension body 430 may be constructed of jersey fabric and/or elastane, but is not limited thereto.

The tension body 430 may be disposed on at least a portion of a second end of the first section 421a, a second end of the second section 421c, a second end of the first section 422a, and/or a second end of the second section 422c. More specifically, the tension body 430 may be disposed between the second end of the first section 421a and/or the second end of the second section 421c. Similarly, the tension body 430 may be disposed between the second end of the first section 422a and/or the second end of the second section 422c. Moreover, the first section 421a, the second section 421c, the first section 422a, and/or the second section 422c may form a cross section (e.g., X-shape) at the tension body 430, such that the cross section increases support with respect to a front portion of the first section 421a and/or the first section 422a. Furthermore, the tension body 430 may have a predetermined tension level to pull the first section 421a, the second section 421c, the first section 422a, and/or the second section 422c toward the tension body 430. In other words, the tension body 430 may be constructed to create a tension level on the shoulder strap assembly 420 while worn, such that the tension body 430 improves a posture of the user 30 while wearing the shoulder strap assembly 420 by forcing the shoulder strap assembly 420 to move toward the tension body 430. Accordingly, the tension body 430 may force the posture of the user 30 to be straight instead of slouched and/or bending forward toward the stomach.

The lumbar support padding 440 may be constructed of foam and/or a gel cushion, but is not limited thereto.

The lumbar support padding 440 may be disposed within at least a portion of the rear section 412. The lumbar support padding 440 may be firmer and/or more rigid, such that the rear section 412 may be firmer and/or more rigid with respect to the front section 411. As such, the lumbar support padding 440 may prevent the back of the user 30 from bending while the rear section 412 is connected to the back of the user 30.

The packet receiving compartment 450 may be constructed of polyethylene terephthalate (PET), closed-cell extruded polystyrene foam, polyurethane, polypropylene, and/or refrigerant gel, but is not limited thereto.

The packet receiving compartment 450 may be disposed on at least a portion of an outer surface of the rear section 412 and/or an inner surface of the rear section 412. The packet receiving compartment 450 may receive a heat pack 10 and/or a cool pack 20 therein. Thus, the packet receiving compartment 450 may be insulated and designed to retain heat from the heat pack 10 and/or cool air from the cold pack 20 therein. Accordingly, the packet receiving compartment 450 may comfort the back of the user 30 in response to dissipating a hot temperature (i.e., increasing a temperature level) and/or a cold temperature (i.e., decreasing the temperature level) on the outer surface and/or the inner surface of the rear section 412.

The temperature adjustment unit 460 may include a mesh of heating coils, a condenser, a fan, and a coolant/refrigerant, but is not limited thereto.

The temperature adjustment unit 460 may be disposed within at least a portion of the rear section 412. Thus, the temperature adjustment unit 460 may increase a temperature level and/or decrease a temperature level of a surface of the rear section 412. More specifically, the temperature adjustment unit 460 may generate heat from the mesh of heating coils and/or generate cold air from the condenser, the fan, and/or the coolant/refrigerant to adjust the temperature level of the surface of the rear section 412. Accordingly, the temperature adjustment unit 460 may comfort the back of the user 30 in response to dissipating a hot temperature (i.e., increasing the temperature level) and/or a cold temperature (i.e., decreasing the temperature level) on the outer surface and/or the inner surface of the rear section 412.

The control unit 470 may include a processing unit 471, a communication unit 472, and a storage unit 473, but is not limited thereto.

The processing unit 471 (or central processing unit, CPU) may include electronic circuitry to carry out instructions of a computer program by performing basic arithmetic, logical, control and input/output (I/O) operations specified by the instructions. The processing unit 471 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit 471 may also include a microprocessor and a microcontroller.

The communication unit 472 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), radio frequency (RF) communication, USB, global positioning system (GPS), Firewire, and Ethernet.

The storage unit 473 may include a random access memory (RAM), a read-only memory (ROM), a hard disk, a flash drive, a database connected to the Internet, cloud-based storage, Internet-based storage, or any other type of storage unit.

The storage unit 473 may be preprogrammed and/or store a software application and/or a mobile application to be executed using the processing unit 471. For ease of description, the software and/or the mobile application will be hereinafter referred to as an app.

The input unit 480 may include a power button 481, a heating button 482, and a cooling button 483, but is not limited thereto.

Although the input unit 480 is described as using a plurality of buttons, the input unit 480 may include a plurality of switches, a plurality of levers, a plurality of knobs, a plurality of knobs, and/or any combination thereof, but is not limited thereto.

The input unit 480 may be disposed on at least a portion of the main body 410 and/or the shoulder strap assembly 420 and connected to the temperature adjustment unit 460. For example, referring again to FIG. 3, the input unit 480 may be disposed on at least a portion of the first section 421a of the first shoulder strap 421. As such, the input unit 480 may be accessible on a front portion of the user 30, such as a chest of the user 30 while wearing the shoulder strap assembly 420. The input unit 480 may correspond to an operation of the temperature adjustment unit 460.

More specifically, the temperature adjustment unit 460 may turn on in response to depressing the power button 481 a first time. Conversely, the temperature adjustment unit 460 may turn off in response to depressing the power button 481 a second time.

It is important to note that the processing unit 471 may communicate with the input unit 480 either through a direct wired connection and/or wirelessly through the communication unit 472, such as Bluetooth. The temperature adjustment unit 460 may increase the temperature level of the rear section 412 in response to depressing the heating button 482. Alternatively, the temperature adjustment unit 460 may decrease the temperature level of the rear section 412 in response to depressing the cooling button 483. Moreover, the processing unit 471 executing the app may determine various temperature levels based on the app in response to depressing the heating button 482 and/or the cooling button 483. Moreover, the processing unit 471 executing the app may save at least one first preferred temperature level of the user 30 in response to depressing the power button 481 and the heating button 482 simultaneously and/or for a predetermined amount of time. Alternatively, the processing unit 471 executing the app may save at least one second preferred temperature level of the user 30 in response to depressing the power button 481 and the cooling button 483 simultaneously and/or for the predetermined amount of time. As such, the processing unit 261 executing the app may automatically set the temperature adjustment unit 250 to adjust the temperature level of the rear section 412 to the at least one first preferred temperature level or the at least one second preferred temperature level after the temperature adjustment unit 460 is next turned on.

Furthermore, the temperature adjustment unit 460 may cycle through a variety of temperature levels in response to depressing the heating button 482 and the cooling button 483 simultaneously, such that the user 30 experiences both hot and/or cold temperatures.

The power source 490 may include a battery (e.g., alkaline, nickel cadmium, lithium ion) and a solar cell, but is not limited thereto.

The power source 490 may be disposed within at least a portion of the rear section 412. The power source 490 may send power to the temperature adjustment unit 460, the control unit 470, and/or the input unit 480.

Therefore, the wearable torso support device 400 may improve posture of the user 30 and/or prevent back pain due to being overweight and/or a pregnancy. Also, the wearable torso support device 400 may alleviate strain on the legs, ankles, and/or feet of the user 30.

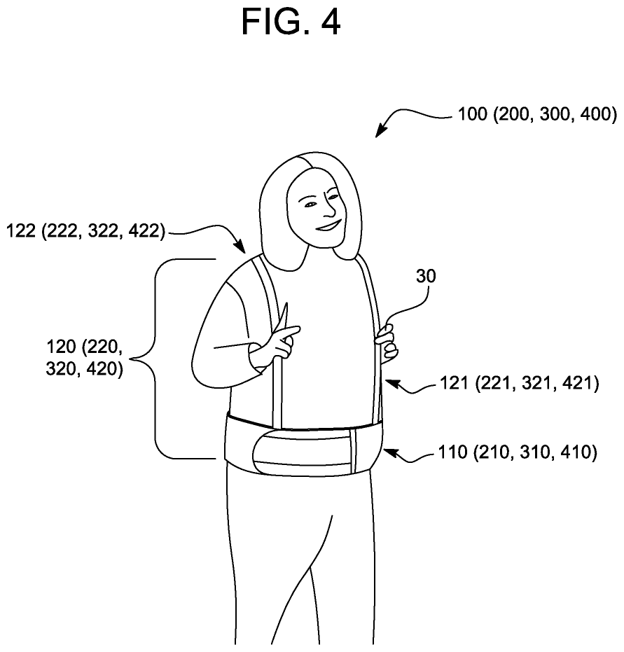

FIG. 4 illustrates a side perspective view of the wearable torso device 100 as disposed on a user 30, according to an exemplary embodiment of the present general inventive concept.

Referring to FIG. 4, the wearable torso device 100 is illustrating showing the main body 110 providing support to the user 30 and the shoulder strap assembly 120 prior to attachment to the main body 110.

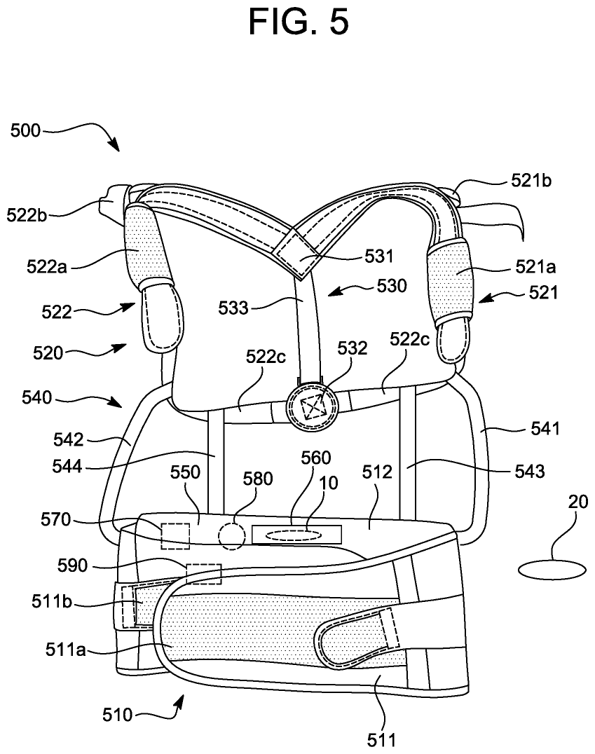

FIG. 5 illustrates a front perspective view of a wearable torso support device 500, according to another exemplary embodiment of the present general inventive concept.

The wearable torso support device 500 may be constructed from at least one of fabric, cloth, plastic, metal, and rubber, etc., but is not limited thereto.

Referring to FIG. 5, the wearable torso support device 500 may include a main body 510, a shoulder strap assembly 520, a tension assembly 530, a shoulder strap connector assembly 540, a lumbar support padding 550, a packet receiving compartment 560, a pressure dissemination unit 570, a power button 580, and a power source 590, but is not limited thereto.

Referring again to FIG. 5, the main body 510 is illustrated to be a waist strap. However, the main body 510 may be a belt, a cord, a back brace, a chain, and/or any other type of waist worn accessory known to one of ordinary skill in the art. Moreover, the main body 510 may be removably connected around a waist and/or a pelvis of a user 30. Moreover, the main body 510 may be worn around the waist and/or the pelvis to cover the waist and/or the pelvis, and support an abdomen and/or a stomach of the user 30. Also, the main body 510 may be constructed of various sizes (e.g., small, medium, large) based on a preference of the user 30. Alternatively, the main body 510 may be a one-size fits all.

The main body 510 may include a front section 511 and a rear section 512, but is not limited thereto.

The front section 511 may be constructed of jersey fabric and/or elastane, but is not limited thereto. The front section 511 may at least partially deform (i.e., stretch, expand, bend) in response to receiving at least a portion of the abdomen and/or the stomach thereon. Additionally, the front section 511 may cover the stomach of the user 30. Also, the front section 511 may prevent the abdomen and/or the stomach from sagging (e.g., dropping toward a ground surface), such that the front section 511 may support the abdomen and/or the stomach of the user 30 while providing comfort.

The front section 511 may include a first body fastener 511a and a second body fastener 511b, but is not limited thereto.

Referring again to FIG. 5, the first body fastener 511a and/or the second body fastener 511b is illustrated to be a hook and loop fastener. However, the first body fastener 511a and/or the second body fastener 511b may include a clip, a clamp, a clasp, a hook, a ratchet, an adhesive (e.g., tape, glue), a magnet, and/or any combination thereof, but is not limited thereto.

The first body fastener 511a and/or the second body fastener 511b may be disposed on at least a portion of the front section 511 and/or the rear section 512. The first body fastener 511a may removably connect to at least a portion of the second body fastener 511b, such that the front section 511 may be separated into two portions to facilitate wearing on the user 30. In other words, the front section 511 may be separated and subsequently connected using the first body fastener 511a and/or the second body fastener 511b to cover the waist of the user 30 and support the stomach of the user 30.

The rear section 512 may be disposed on at least a portion of each end of the front section 511. In other words, the rear section 512 may comprise half of a size of the main body 510 and the front section 511 may comprise half of the size of the main body 510. The rear section 512 may cover at least a portion of a back of the user 30.

The shoulder strap assembly 520 may include a first shoulder strap 521 and a second shoulder strap 522, but is not limited thereto.

The first shoulder strap 521 may include a first section 521a, a first shoulder strap fastener 521b, and a second section 521c, but is not limited thereto.

The first section 521a of the first shoulder strap 521 may be connected to at least a portion of the main body 510.

Referring again to FIG. 5, the first shoulder strap fastener 521b is illustrated to be a buckle. However, the first shoulder strap fastener 521b may be hooks and loops, a twine, a string, a rope, a magnet, a clip, a clamp, a clasp, a hook, and/or any combination thereof, but is not limited thereto.

The first shoulder strap fastener 521b may adjust a length of the first section 521a, such as increasing the length of the first section 521a by moving the first section 521a through the first shoulder strap fastener 521b in a first direction (e.g., toward the front section 511), and decreasing the length of the first section 521a by moving the first section 521a through the first shoulder strap fastener 521b in a second direction (e.g., away from the front section 511) opposite with respect to the first direction.

The second section 521c of the first shoulder strap 521 may be connected to at least a portion of the main body 510.

The second shoulder strap 522 may include a first section 522a, a second shoulder strap fastener 522b, and a second section 122c, but is not limited thereto.

The first section 522a of the second shoulder strap 522 may be connected to at least a portion of the main body 510.

Referring again to FIG. 5, the second shoulder strap fastener 522b is illustrated to be a buckle. However, the second shoulder strap fastener 522b may be hooks and loops, a twine, a string, a rope, a magnet, a clip, a clamp, a clasp, a hook, and/or any combination thereof, but is not limited thereto.

The second shoulder strap fastener 522b may adjust a length of the first section 522a, such as increasing the length of the first section 522a by moving the first section 522a through the second shoulder strap fastener 522b in a first direction (e.g., toward the front section 511), and decreasing the length of the first section 522a by moving the first section 522a through the second shoulder strap fastener 522b in a second direction (e.g., away from the front section 511) opposite with respect to the first direction.

The second section 522c of the second shoulder strap 522 may be connected to at least a portion of the main body 510.

As such, the shoulder strap assembly 520 may by adjusted based on the preference of the user 30. Also, the shoulder strap assembly 520 may be connected to shoulders of the user 30, such that the main body 510 may be suspended from the shoulder strap assembly 520 while being worn by the user 30.

The tension assembly 530 may be constructed of jersey fabric and/or elastane, but is not limited thereto.

The tension assembly 530 may include a first tension body 531, a second tension body 532, and a body connecting strap 533, but is not limited thereto.

The first tension body 531 may be disposed on at least a portion of a second end of the first section 521a and/or a second end of the first section 522a. More specifically, the first tension body 531 may be disposed between the second end of the first section 521a and/or the second end of the first section 522a. Similarly, the tension body 532 may be disposed on at least a portion of a second end of the second section 521c and/or a second end of the second section 522c, such that the second tension body 532 may be disposed between the second end of the second section 521c and/or the second end of the second section 522c.

Moreover, the body connecting strap 533 may connect the first tension body 531 to the second tension body 532. As such, the first tension body 531, the second tension body 532, and/or the body connecting strap 533 may increase support with respect to a front portion of the first section 521a and/or the first section 522a. Furthermore, the tension assembly 530 may have a predetermined tension level to pull the first section 521a, the second section 521c, the first section 522a, and/or the second section 522c toward the tension assembly 530. In other words, the tension assembly 530 may be constructed to create a tension level on the shoulder strap assembly 520 while worn, such that the tension assembly 530 improves a posture of the user 30 while wearing the shoulder strap assembly 520 by forcing the shoulder strap assembly 520 to move toward the tension assembly 530. Accordingly, the tension assembly 530 may force the posture of the user 30 to be straight instead of slouched and/or bending forward toward the stomach.

The shoulder strap connector assembly 540 may include a first front strap 541, a second front strap 542, a first rear strap 543, and a second rear strap 544, but is not limited thereto.

The first front strap 541 may connect the first section 521a of the first shoulder strap 521 to at least a portion of the front section 511. Similarly, the second front strap 542 may connect the first section 522a of the second shoulder strap 522 to at least a portion of the front section 511. Moreover, the first front strap 541 may be disposed a first distance away from the second front strap 542.

The first rear strap 543 may connect the second section 521c of the first shoulder strap 521 to at least a portion of the rear section 512. Similarly, the second rear strap 544 may connect the second section 522c of the second shoulder strap 522 to at least a portion of the rear section 512. Moreover, the first rear strap 543 may be disposed a second distance away from the second rear strap 544.

As such, the main body 510 may be suspended from the shoulder strap connector assembly 540 at a third distance away from the shoulder strap assembly 520. It is important to note that the shoulder strap connector assembly 540 may increase flexibility of the user 30 while wearing the main body 510 and/or the shoulder strap assembly 520. More specifically, the shoulder strap connector assembly 540 may at least partially deform (i.e., bend, stretch, expand) in response to movement of the main body 510 in a different direction with respect to movement of the shoulder strap assembly 520.

The lumbar support padding 550 may be constructed of foam and/or a gel cushion, but is not limited thereto.

The lumbar support padding 550 may be disposed within at least a portion of the rear section 512. The lumbar support padding 550 may be firmer and/or more rigid, such that the rear section 512 may be firmer and/or more rigid with respect to the front section 511. As such, the lumbar support padding 550 may prevent the back of the user 30 from bending while the rear section 512 is connected to the back of the user 30.

The packet receiving compartment 560 may be constructed of polyethylene terephthalate (PET), closed-cell extruded polystyrene foam, polyurethane, polypropylene, and/or refrigerant gel, but is not limited thereto.

The packet receiving compartment 560 may be disposed on at least a portion of an outer surface of the rear section 512 and/or an inner surface of the rear section 512. The packet receiving compartment 560 may receive a heat pack 10 and/or a cool pack 20 therein. Thus, the packet receiving compartment 560 may be insulated and designed to retain heat from the heat pack 10 and/or cool air from the cold pack 20 therein. Accordingly, the packet receiving compartment 560 may comfort the back of the user 30 in response to dissipating a hot temperature (i.e., increasing a temperature level) and/or a cold temperature (i.e., decreasing the temperature level) on the outer surface and/or the inner surface of the rear section 512.

The pressure dissemination unit 570 may be disposed within at least a portion of the rear section 512. The pressure dissemination unit 570 may include various devices to simulate a massage, including, but not limited to, vibration devices, pistons, mechanical rollers, compression devices, stretching devices, kneading devices, pressing devices, oscillators, gears, etc. As such, the pressure dissemination unit 570 may provide a pressured sensation to the back of the user 30, thus simulating a massage.

The power button 580 may be disposed on at least a portion of the rear section 512 and connected to the pressure dissemination unit 570. The pressure dissemination unit 570 may turn on in response to depressing the power button 580 a first time. Conversely, the pressure dissemination unit 570 may turn off in response to depressing the power button 580 a second time.

Alternatively, the power button 580 may be a control panel 580 to change at least one operation of the pressure dissemination unit 570, such as a speed, pressure intensity, or pre-programmed massage style, such as Swedish, deep tissue, shiatsu, Thai, simulation of fingers, elbows, hands chopping, vibration, reiki, reflexology, and/or pounding.

The power source 590 may include a battery (e.g., alkaline, nickel cadmium, lithium ion) and a solar cell, but is not limited thereto.

The power source 590 may be disposed within at least a portion of the rear section 512. The power source 590 may send power to the pressure dissemination unit 570 and/or the power button 580.

Therefore, the wearable torso support device 500 may improve posture of the user 30 and/or prevent back pain due to being overweight and/or a pregnancy. Also, the wearable torso support device 500 may alleviate strain on the legs, ankles, and/or feet of the user 30.

Furthermore, the depictions between the FIGS. 1A-5 may differ based on various versions of the present general inventive concept, or the preference of the user 30. For example, the rear section 112 is convex shaped and rounded whereas the rear section 212 has a dome shape and the rear section 512 is planar. Also, referring again to FIGS. 1A-5, the main body 510 may have a width greater than a width of the main body 110 to provide more support to the user. Similarly, the shoulder strap assembly 120, the shoulder strap assembly 220, the shoulder strap assembly 320, and/or the shoulder strap assembly 420 may differ with respect to the shoulder strap assembly 520 in size (e.g., width, length) based on the preference of the user. For example, the width of the shoulder strap assembly 520 may be greater than the width of the shoulder strap assembly 120, the shoulder strap assembly 220, the shoulder strap assembly 320, and/or the shoulder strap assembly 420, such that the shoulder strap assembly 520 may be accessible for people with a height and/or weight greater than a predetermined height and/or a predetermined weight. Conversely, the shoulder strap assembly 120, the shoulder strap assembly 220, the shoulder strap assembly 320, and/or the shoulder strap assembly 420 may be accessible for people with the height and/or the weight less than the predetermined height and/or the predetermined weight.

The present general inventive concept may include a wearable torso support device 100, including a main body 110 to removably connect around a pelvis of a user 30, including a front section 111 to receive and support a stomach of the user 30 thereon, a rear section 112 disposed on at least a portion of each end of the front section 111 to support at least a portion of a back of the user 30, and a plurality of body fasteners 113 removably connected to at least a portion of at least one of the front section 111 and the rear section 112, and a shoulder strap assembly 120 to suspend the main body 110 from shoulders of the user 30 while being worn, the shoulder strap assembly 120 including a first shoulder strap 121 disposed on at least a portion of the main body 110 to removably connect to at least one of the plurality of body fasteners 113 and is adjustable in length based on a movement of the first shoulder strap 121, and a second shoulder strap 122 disposed on at least a portion of the main body 110 to removably connect to another at least one of the plurality of body fasteners 113 and is adjustable in length based on a movement of the second shoulder strap 122.

The first shoulder strap 121 and the second shoulder strap 122 each may include a first section 121a (122a) to removably connect to at least one of the plurality of body fasteners 113, a shoulder strap fastener 121b (122b) disposed on at least a portion of the first section 121a (122a) to increase a length of the first section 121a (122a) in response to moving the first section 121a (122a) through the shoulder strap fastener 121b (122b) in a first direction, and decrease the length of the first section 121a (122a) in response to moving the first section 121a (122a) through the shoulder strap fastener 121b (122b) in a second direction opposite with respect to the first direction, and a second section 121c (122c) disposed on at least a portion of the main body 110 and connected to the first section 121a (122a).

The wearable torso support device 100 may further include a tension body 130 disposed between the first section 121a (122a) and the second section 121c (122c) to create a tension level on the shoulder strap assembly 120 while worn, such that the tension body 130 forces the shoulder strap assembly 120 to move toward the tension body 130.

The wearable torso support device 100 may further include a lumbar support padding 140 disposed within at least a portion of the rear section 112 to prevent the back of the user 30 from bending while the rear section 112 is connected to the back of the user 30.

The lumbar support padding 140 may make the rear section 112 more rigid than the front section 111.

The wearable torso support device 100 may further include a packet receiving compartment 150 disposed on at least a portion of the main body 110 to receive at least one of a heat pack 10 and a cool pack 20 therein, such that the packet receiving compartment 150 increases a temperature level or decreases the temperature level of the main body 110.

The wearable torso support device 100 may further include a pressure dissemination unit 160 disposed within at least a portion of the main body 110 to simulate a massage.

The wearable torso support device 200 may further include a temperature adjustment unit 250 disposed within at least a portion of the main body 210 to increase a temperature level or decrease the temperature level of the main body 210 using at least one of a mesh of heating coils, a condenser, a fan, and a refrigerant to adjust the temperature level of the main body 210.

The wearable torso support device 100 may further include a control unit 260 comprising a processing unit 261 running a program thereon to determine the temperature level of the temperature adjustment unit 250.

The wearable torso support device 100 may further include an input unit 270 comprising at least one button, the input unit 270 disposed on at least a portion of the shoulder strap assembly 220 to adjust the temperature level of the temperature adjustment unit 250 using the control unit 260 in response to depressing at least one button of the input unit 270.

The processing unit 261 may save at least one preferred temperature level in response to depressing the at least one button for a predetermined amount of time.

The processing unit 261 may automatically set the temperature adjustment unit 250 to adjust the temperature level to at least one preferred temperature level.

The wearable torso support device 300 may further include an external controller 370 removably connected to at least a portion of the main body 310 to transmit a command to the control unit 360 to adjust the temperature level of the temperature adjustment unit 350 in response to the external controller 370 receiving at least one input.

The external controller 370 may include a display unit 372 to display an image of the rear section 312 and activate at least one portion of the temperature adjustment unit 350 corresponding to the portions of the rear section 312 that have been selected on the display unit 372.

The present general inventive concept may also include a wearable torso support device 400, including a main body 410 to removably connect around a pelvis of a user and support a stomach of the user thereon, a shoulder strap assembly 420 disposed on at least a portion of the main body 410 to suspend the main body 410 from shoulders of the user 30 while being worn, such that the shoulder strap assembly 420 is adjustable in length using a ratchet, a packet receiving compartment 450 disposed on at least a portion of the main body 410 to receive at least one of a heat pack 10 and a cool pack 20 therein, such that the packet receiving compartment 450 increases a temperature level or decreases the temperature level of the main body 410, and a temperature adjustment unit 460 disposed within at least a portion of the main body 410 to increase a temperature level or decrease the temperature level of the main body 410 using at least one of a mesh of heating coils, a condenser, a fan, and a refrigerant to adjust the temperature level of the main body 410.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A wearable torso support device, comprising:
a main body to removably connect around a pelvis of a user, comprising:
a front section to receive and support a stomach of the user thereon,
a rear section disposed on at least a portion of each end of the front section to support at least a portion of a back of the user, and
a plurality of body fasteners removably connected to at least a portion of at least one of the front section and the rear section; and
a shoulder strap assembly to suspend the main body from shoulders of the user while being worn, the shoulder strap assembly comprising:
a first shoulder strap disposed on at least a portion of the main body to removably connect to at least one of the plurality of body fasteners and is adjustable in length based on a movement of the first shoulder strap, and
a second shoulder strap disposed on at least a portion of the main body to removably connect to another at least one of the plurality of body fasteners and is adjustable in length based on a movement of the second shoulder strap.

2. The wearable torso support device of claim 1, wherein the first shoulder strap and the second shoulder strap each comprise:
a first section to removably connect to at least one of the plurality of body fasteners;
a shoulder strap fastener disposed on at least a portion of the first section to increase a length of the first section in response to moving the first section through the shoulder strap fastener in a first direction, and decrease the length of the first section in response to moving the first section through the shoulder strap fastener in a second direction opposite with respect to the first direction; and
a second section disposed on at least a portion of the main body and connected to the first section.

3. The wearable torso support device of claim 2, further comprising:
a tension body disposed between the first section and the second section to create a tension level on the shoulder strap assembly while worn, such that the tension body forces the shoulder strap assembly to move toward the tension body.

4. The wearable torso support device of claim 1, further comprising:
a lumbar support padding disposed within at least a portion of the rear section to prevent the back of the user from bending while the rear section is connected to the back of the user.

5. The wearable torso support device of claim 4, wherein the lumbar support padding makes the rear section more rigid than the front section.

6. The wearable torso support device of claim 1, further comprising:
a packet receiving compartment disposed on at least a portion of the main body to receive at least one of a heat pack and a cool pack therein, such that the packet receiving compartment increases a temperature level or decreases the temperature level of the main body.

7. The wearable torso support device of claim 1, further comprising:
a pressure dissemination unit disposed within at least a portion of the main body to simulate a massage.

8. The wearable torso support device of claim 1, further comprising:
a temperature adjustment unit disposed within at least a portion of the main body to increase a temperature level or decrease the temperature level of the main body using at least one of a mesh of heating coils, a condenser, a fan, and a refrigerant to adjust the temperature level of the main body.

9. The wearable torso support device of claim 8, further comprising:
a control unit comprising a processing unit running a program thereon to determine the temperature level of the temperature adjustment unit.

10. The wearable torso support device of claim 9, further comprising:
an input unit comprising at least one button, the input unit disposed on at least a portion of the shoulder strap assembly to adjust the temperature level of the temperature adjustment unit using the control unit in response to depressing at least one button of the input unit.

11. The wearable torso support device of claim 10, wherein the processing unit saves at least one preferred temperature level in response to depressing the at least one button for a predetermined amount of time.

12. The wearable torso support device of claim 9, wherein the processing unit automatically sets the temperature adjustment unit to adjust the temperature level to at least one preferred temperature level.

13. The wearable torso support device of claim 9, further comprising:
an external controller removably connected to at least a portion of the main body to transmit a command to the control unit to adjust the temperature level of the temperature adjustment unit in response to the external controller receiving at least one input.

14. The wearable torso support device of claim 12, wherein the external controller comprises:
a display unit to display an image of the rear section and activate at least one portion of the temperature adjustment unit corresponding to the portions of the rear section that have been selected on the display unit.

15. A wearable torso support device, comprising:
a main body to removably connect around a pelvis of a user and support a stomach of the user thereon;
a shoulder strap assembly disposed on at least a portion of the main body to suspend the main body from shoulders of the user while being worn, such that the shoulder strap assembly is adjustable in length using a ratchet;
a packet receiving compartment disposed on at least a portion of the main body to receive at least one of a heat pack and a cool pack therein, such that the packet receiving compartment increases a temperature level or decreases the temperature level of the main body; and
a temperature adjustment unit disposed within at least a portion of the main body to increase a temperature level or decrease the temperature level of the main body using at least one of a mesh of heating coils, a condenser, a fan, and a refrigerant to adjust the temperature level of the main body.
